(19) United States
(12) Patent Application Publication
(10) Pub. No.: US 2023/0124323 A1
(43) Pub. Date: Apr. 20, 2023

(54) Optimization of Radionuclides for Treatment of Cutaneous Lesions

(71) Applicant: Kenneth Wesley Brooks, Windermere, FL (US)

(72) Inventor: Kenneth Wesley Brooks, Windermere, FL (US)

(21) Appl. No.: 17/503,350

(22) Filed: Oct. 17, 2021

(51) Int. Cl. A61N 5/10

(52) U.S. Cl. CPC A61N 5/1029; A61N 2005/1089; A61N 2005/1094; A61N 2005/1091

(57) ABSTRACT

The present invention provides a radioactive patch comprising a layer of a mixture of a radionuclide with a nonreactive adhesive agent coated thereon in the form of a tape, and a laminating layer, wherein the patch comprises, a high Z shielding layer placed on the opposing side of the patch away from the patient tissue, and comprising at least one of: lead, tungsten, iron, silver, gold, platinum, copper, brass; and wherein the patch comprises, a low Z shielding layer comprising at least one of: teflon, pma, pvc, lucite, boron carbide, graphite, carbon fiber, bakelite; and wherein the radionuclide comprises at least one of: Y-90, Ho-166, LU-166, I-125, PD-103, LU-166, or any combination thereof.

### REFERENCES CITED

### Other Publications

1 https://www.world-nuclear.org/information-library/non-power-nuclear-applications/radioisotopes-research/radioisotopes-in-medicine.aspx, Accessed October 2021.
2. https://www.aad.org/media/stats-skin-cancer, Accessed October 2021.
3. J. D. Lee et al, Radionuclide Therapy of Skin Cancers and Bowen's Disease Using a Specially Designed Skin Patch, The Journal of Nuclear Medicine, Vol. 38 No. 5. May 1997.
4. K. B. Park et al, U.S. Pat. No. 5,871,708, Feb. 16, 1999. Definitive and Postoperative Radiation Therapy for Basal and Squamous Cell Cancers of the Skin: Executive Summary of the American Society for Radiation Oncology, Clinical Proactive Guideline.
5. A. Likhacheva, et al, Definitive and Postoperative Radiation Therapy for Basal and Squamous Cell Cancers of the Skin: Executive Summary of the American Society for Radiation Oncology, Clinical Proactive Guideline, Practical Radiation Oncology, Dec. 9, 2019.
6. John Y. S. Kim, et al, Guidelines of care for the management of basal cell carcinoma, JAAD, Volume 78, Issue 3, p 540-559, Mar. 1, 2018.
7 John Y.S. Kim, et al, Guidelines of care for the management of cutaneous squamous cell carcinoma, JAAD, Volume 78, Issue 3, p 540-559, Mar. 1, 2018.
8. U.S. Pat. No. 5,871,708 Issued Feb. 16, 1999 to Park, et. al.
9. https://www.energy.gov/articles.department-energy-provide-16-million-isotope-production-rd.
10. https://www.novartis.com/news/media-releases/novartis-completes-tender-offer-advanced-accelerator-applications-sa-and-announces-commencement-subsequent-offering-period.

### BACKGROUND OF THE INVENTION

### 1. Field of the Invention

The present invention relates to the technical field of radiation therapy and specifically within this radionuclide therapy in the form of a superficial apparatus or a patch used to externally deliver radiation dose in the form of energetic photons and electrons emanating from radioactive elements and the optimal configuration thereof. In particular, the present invention relates to a small superficial apparatus which contains the optimal mixture of radioisotopes and is attached to the surface of the skin or other body part to treat lesions in each range with a given mixture of isotopes. The present invention also relates to the process of using the information about the lesion and novel isotopes to 1) selectively design a mixture of isotopes whose radiation decay processes can provide full dose coverage of the lesion, and 2) based upon the mixture of the isotopes, also choose a thickness of material comprising the interface between the lesion and the radiation source to give a radiological bolus effect and further 3) to design the back side or outward facing portions of the patch to contain proper shielding for the timeframe and anticipated exposure risks of the patient or any proximal persons in contact with the patient during treatment.

### 2. Description of the Related Art

Radionuclides have been used in medicine from early in the history of their discovery at the turn of the 20th century. Radionuclides as referred to here are either excited by-products from nuclear fission whose chemical properties place them within the lanthanides in the periodic table of the elements or are specially created targets placed in a high neutron or photon flux of a nuclear reactor or high energy particle accelerator. The irradiation of the targets allows the selected elements within the target to transmute from the energy imparted by the neutrons or photons present ultimately causing them to transmute into a medically useful isotope. Regardless of how they are created, they are unstable and decay by either alpha particle, beta particle or photons or some combination of all three and can create a chain of decay particles and daughter products. In some cases, special isotopes decay with particles and energies which are suitable for use with many diseases in the human body. [1]

Originally produced from nuclear reactor fission products isolated chemically and prepared for medical use, the production was largely confined to nuclear reactors with specialized processing. However with the advancement of particle accelerators becoming more compact and more cost effective than a nuclear reactor, radiopharmaceuticals for diagnostic and therapeutic uses have become reliable as a safe and steady supply of certain workhorse isotopes such Technetium-99 for cardiac imaging, Iodine-131 bound into a monoclonal antibody drug used for Thyroid treatment and Flourine-18 for producing Fluorodeoxyglucose (FDG) for Positron Emission Tomography (PET) scanning for cancer assessment as just a few of the high volume examples coming into mainstream all over the world over the last several decades. [1]

Accordingly, what is needed in the art is an efficient means to take advantage of the range of radionuclide isotopes available for treatment of cutaneous lesions in the human body. Cutaneous lesions may take the form of malignancies such as melanoma or non-melanoma basal cell and squamous cell carcinomas, lymphomas, and several other skin lesions which require treatment. The apparatus should provide a ready means to deliver particles with very high energies emanating from radionuclide isotopes, which may be newly discovered or well established as suitable for use with many diseases in the human body. Also needed is a methodology for the selection of the Radionuclide isotope or combinations of Radionuclide isotopes for use within the apparatus for patient treatment of a given cutaneous lesion. It is thus to such apparatus and method that the present invention is primarily directed.

### SUMMARY OF THE INVENTION

The disadvantages of the prior art are overcome by the present invention which, in one aspect, is a radioactive patch comprising a carrier, a layer of a mixture of a radionuclide with a nonreactive adhesive agent coated thereon in the form of a tape, and a laminating layer, and the patch comprising a high Z shielding layer placed on the opposing side of the patch away from the patient tissue, and the high Z layer comprising at least one of: lead, tungsten, iron, silver, gold, platinum, copper, brass. The patch also comprises a low Z shielding layer of comprising at least one of: teflon, pma, pvc, lucite, boron carbide, graphite, carbon fiber, bakelite; and the radionuclide comprises at least one of: Y-90, Ho-166, LU-166, I-125, PD-103, LU-166, or any combination thereof.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE DRAWINGS

FIG. 1 is a perspective view of a cutaneous lesion as shown on a human forearm.

FIG. 2 is a perspective view of a cutaneous lesion as shown on a human forehead.

FIG. 3A depicts a table of Electron and Photon Emitting Radionuclides compared to 6 MeV electrons and 100 kVp X-ray sources for treating cutaneous lesions.

FIG. 3B depicts a graphical depth dose comparison of commonly used Xray & Gamma Ray sources.

FIGS. 4A, 4B, and 4C compare Ho-166 to Y-90 and Lu-166 in terms of decay spectra of electrons.

FIG. 5A depicts a radionuclide patch design which allows dosimetry optimization.

FIG. 5B depicts a radionuclide patch design which allows dosimetry optimization to be custom shaped for a given body site.

FIG. 6A-6B depicts a radionuclide patch design which allows dosimetry optimization via a flexible patch design shaped for large areas as well as contour changes.

### DETAILED DESCRIPTION OF THE INVENTION

The present invention provides an efficient means to take advantage of the range of radionuclide isotopes available for treatment of cutaneous lesions of the human body. The apparatus provides a ready means to deliver/expose the patient to particles and energies of radionuclide isotopes via an adhesive patch with radionuclide therein. Also presented is a methodology for the selection of the Radionuclide isotope or combinations of Radionuclide isotopes for use within the apparatus for patient treatment of a given cutaneous lesion.

In the field of radiation therapy, radiation in many forms is directed towards lesions both benign and malignant which benefit from the dose of radiation as determined by a clinician. It is the role of the radiation oncologists, radiation physicists and engineers at device vendors to consider how to best arrange a given set of radiation properties of a radiation source in such a way as to produce a maximal tumor destruction and a minimal normal adjacent tissue destruction. The difference between these two objectives is a simplified definition of a concept known as the ‘therapeutic window’ in radiation therapy. Each new technology is accepted or not based upon the ability of the technique to expand this window for a given condition by creating more destruction within lesions and less damage to normal tissues.

The therapeutic window for cutaneous lesions is well established for traditional delivery mechanisms comprised of large expensive capital devices mostly purchased by large hospitals and medical centers. The point of care where the skin lesion is diagnosed is almost always at a dermatologist office where these large pieces of equipment are typically not present. Certain cohorts of patients are not good surgical candidates and for these a reliable source of treatment which can at least maintain the therapeutic window of large radiation devices but logistically condensed down into a wearable patch with radionuclides is a novel invention and would be a welcome alternative to surgery or weeks of visits to a radiation bunker of a large hospital.

The key issue for the creation of a wearable patch of radionuclides is the ability of the isotope to control the radiation with a favorable side effect profile relative to other radiation and surgical alternatives. With the advent of new isotopes, the field is advancing, and it is a chance to reconsider this approach for the application to cutaneous lesions. The wearable patch of this invention allows for a delivery mechanism for the radiation to be conformed to the shape of the lesion in the superficial plane and then optimized in depth by considering the unique properties of various available isotopes and the clinical condition of a given patient's lesion. The invention also covers optimizing the depth dose curve with a built-in bolus feature which serves to shift in tissue as desired and a back covering that also serves as a safety shield. The object of the invention is to provide this patch device capable of accepting various forms of radioactive substrates containing the nuclides and packaging them into the aforementioned device the details of which will be described in the following.

More uses for radionuclides have arisen from recent innovations in this area introducing a growing list of nuclides enabled by the ability to make these novel isotopes having optimized energy decay properties for certain diseases and delivery technologies produced more conveniently than within a nuclear reactor. As the production and biochemical delivery mechanisms have evolved along with all medical science and radiochemistry over the last 30 years more factors are coming together to enable a more selective and strategic use of isotopes to be integrated into sophisticated macro molecules or otherwise chemically bound to elements which have physiological importance for applying the specific decay scheme energy from the isotope for a given medical condition.

In the United States, the U.S. Department of Energy has been actively supporting the development of advanced accelerators capable of replacing nuclear reactors for these strategically crucial isotopes relied upon to save lives in medicine currently. The result of these concentrated development efforts has companies and established laboratories expanding to develop new technology to address these priorities. One such effort started from the CERN laboratory led to the development of Leuticium-166 in a public company called Advanced Accelerator Applications which was purchased by the Swiss drug company Novartis for $3.5 billion dollars about three years ago now. Since that time a flurry of activity has occurred, and the technical development of special isotope production has risen sharply attempting to meet the demand for these ‘boutique’ isotopes for a given medical condition and/or drug or agent which can deliver it to a lesion.

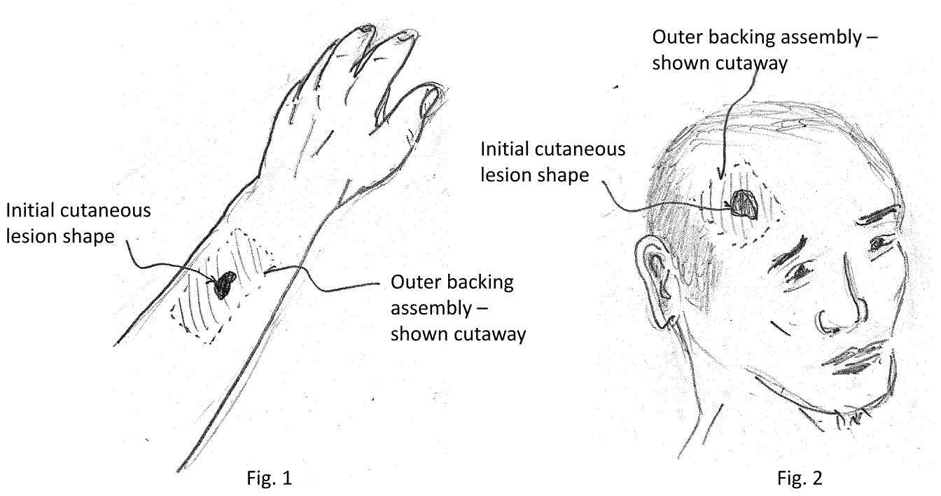

In a first embodiment, this invention relates to the application a ready supply of a wide variety of radionuclides specifically for the treatment of cutaneous lesions. Cutaneous lesions exist within the first centimeter of tissue from the body surface. FIGS. 1, 2 depicts some common sizes, shapes, and locations for lesions though they can appear anywhere on the skin surface, they are mostly apparent on the extremities and exposed portions of face, head and neck. As depicted in FIG. 1, a cutaneous lesion is shown on a human forearm. As depicted in FIG. 2, a cutaneous lesion is shown on a human forehead. The lesions can be benign or cancerous processes and radiation has long been established as an effective curative treatment including both high energy electrons and low energy photons. This invention seeks to replicate the excellent curative and cosmetic benefits established with particle sources from linear accelerators and X-ray tubes but achieved with the use of selective radionuclides alone or in combination.

The motivation for using an isotope instead of a large linear accelerator or even x-ray device designed for therapeutic use has to do with the high prevalence of skin cancer in the developed world, which for reference is cited in literature at approximately 3.5 million patients having over 5.5 million lesions treated annually in the United States alone in 2019 and growing. This compares to around 1.2 million patients for all other solid tumors in the body annually in the United States or somewhere on the order of 5 times as many skin lesions to treat as all other solid tumors in the body in the Unites States. [2] It extrapolates into a very large number of patients on a global scale impacted by this disease annually.

The standard of care involves dermatologists using surgical techniques such a MOHS microsurgery, conventional excision, curettage, destruction, and radiation therapy using megavoltage electrons from linear accelerators at 4 or 6 MeV and x-rays ranging in energy from 50 to 100 kVp. In addition to their being a very large number of lesions to treat annually, using isotopes can allow all sizes of dermatology practices to consider their use on patients which may not be suitable for surgical treatment due to contraindications such as blood thinners and who also may not be able to travel to a clinic from 15 to 30 times for treatments from a linear accelerator or an X-ray system. In the US, large practice groups can consolidate high volumes of patients from their satellite smaller office which are suitable for radiation therapy to help offset the overhead and cost of creating a dedicated skin cancer treatment facility within their practice at each location. Use of a patch that could replace those large machines can effectively democratize the use of radiation therapy, particularly radionuclide therapy, and spare unnecessary or unwanted surgeries all at a smaller logistical impact to the dermatology practices. The necessary technologists, physicists, and licensed isotope prescribing physicians are readily available to properly support such activities in dermatologist's offices at the point of care and diagnosis to avoid patients needing to navigate a large hospital nuclear medicine department or a radiation oncology department. This technology can also enable a truly multidisciplinary approach to provided more optimal care for appropriate portions of the skin cancer patient population.

Such an approach was already conceived using a lower energy isotope Holmium-166. Holmium's decay scheme while reported to be useful in one publication, demonstrated in that publication that the electrons emitted are not penetrating enough to closely replicate a megavoltage electron beam from a linear accelerator for such lesions or cover the same depth as keV energy x-rays [3, 4]. In fact, the published results from the authors reported negative cosmetic sequalae associated with the use of a Ho-166 patch which are consistent with the low energy beta particle depositing their energy within the first 2-3 mm of cutaneous tissue and all within periods of 1-2 hours. The use of Ho-166 for skin lesions was apparently stopped and is still not in widespread use today. This result is apparently partially due to the suboptimal energy of the electrons emitted for use in cutaneous lesions from Ho-166. Very effective surgical techniques and external beam radiation sources give effective control without causing such significant side effects as discussed in the Ho-166 study.

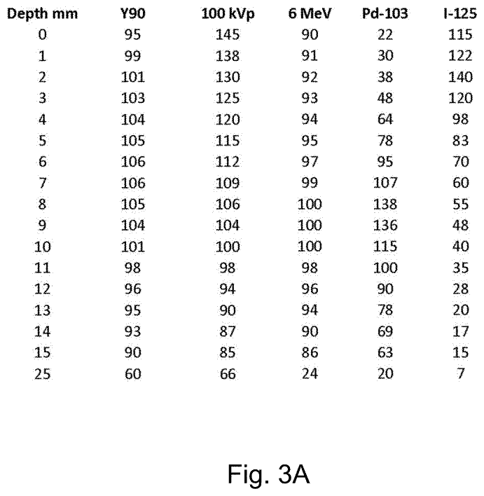

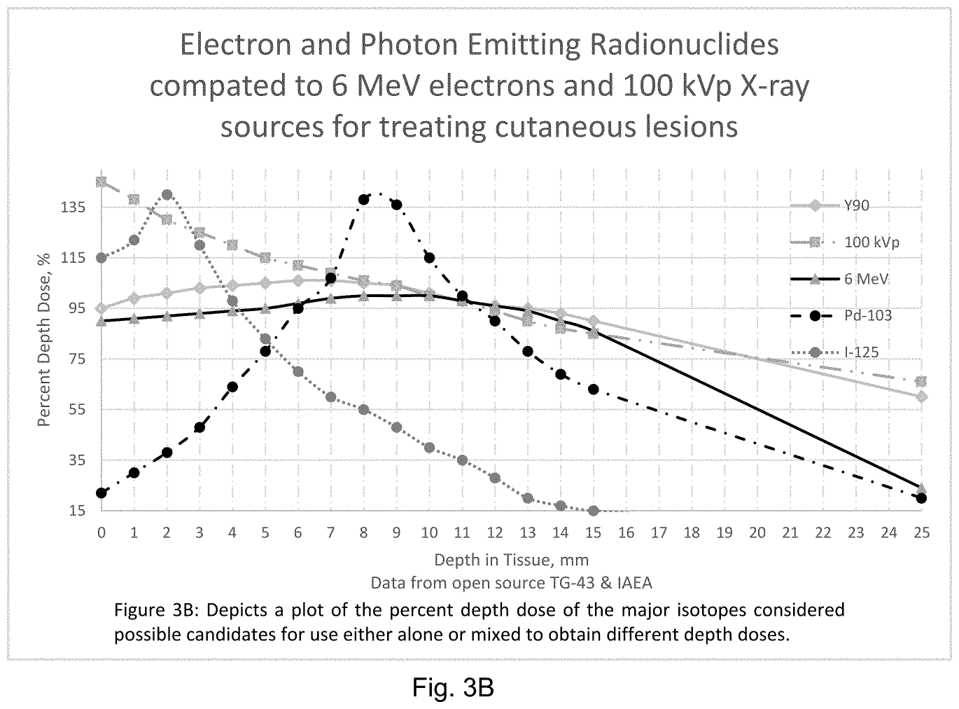

Our studies have shown that using a nuclide such as Yittrium-90, which has become widely established for radioembolization in recent years for cancerous and benign processes that involve excessive vascularity, one might more closely approach a depth dose curve of an electron 4-6 MeV linear accelerator beam or at least achieve something close to a keV x-ray source. FIG. 3A presents a table of Electron and Photon Emitting Radionuclides compared to 6 MeV electrons and 100 kVp X-ray sources for treating cutaneous lesions. FIG. 3B depicts a graphical depth dose comparison of commonly used Xray & Gamma Ray sources. As depicted in FIG. 3B, note how Pd-103 and I-125 bracket the commonly used 100 kVp Xray used in skin cancer clinics worldwide today. Iodine deposits most of its energy within the first centimeter of tissue while palladium is shifted more toward a peak deposition around 1 cm and having better fall off beyond 1 cm. Proper use of a material and technique known in radiotherapy as ‘bolus’ involves the application of a tissue equivalent, gelatinous material of a known thickness which radiologically mimics tissue such that a bolus of a known thickened effectively shifts the percent depth dose curves as the radiation impinges upon the bolus first and the beam begins to deposit the dose at that point instead of the actual skin surface. Usage of a bolus with Pd-103 can affect a shift of the depth dose curve back to any depth desired for a given tumor thickness by applying a bolus material of that thickness to the patient at the beam entry point. This is an example of what is possible in terms of optimizing an isotope applicator for a given isotope and patient just by adding bolus. It is also possible to mix two different isotopes in with some abundance ration which would yield a combined based on the relative abundance of in this example, I-125 and Pd-103.

The combined depth dose curve being some optimal curve to place dose closer to the surface or farther from the surface or more uniform throughout from surface to prescription depth depending upon the clinician's intent. Without mixing, it is apparent from FIG. 3B that Y-90 very closely tracks the depth dose behavior of 6 MeV electrons from a linear accelerator thus it could be considered alone as a solitary isotope in the current invention to fabricate a wearable patch as what was discussed in the Ho-166 references, but with Y-90 in sufficient activity to deliver the dose per fractions similar to what is done for 6 MeV electrons as in linear accelerators.

Non-melanoma skin cancers for example typically do not exhibit excessive vascularity at least in their earlier stages thus radioembolization techniques would not be applicable. These lesions are very well controlled with good cosmetic results using a radiation beam of 4-6 MeV electrons and 75 kVp x-rays as has been described in many publications and in recent guidance by the ASRO (American Society of Radiation Oncology) [5] and the AAD (American Academy of Dermatology. Also, both 4-6 MeV electrons and kilovoltage x-rays have been included in the United States (National Comprehensive Cancer Network) and the equivalent European clinical guidelines for cancer treatment for cutaneous lesions for many decades.

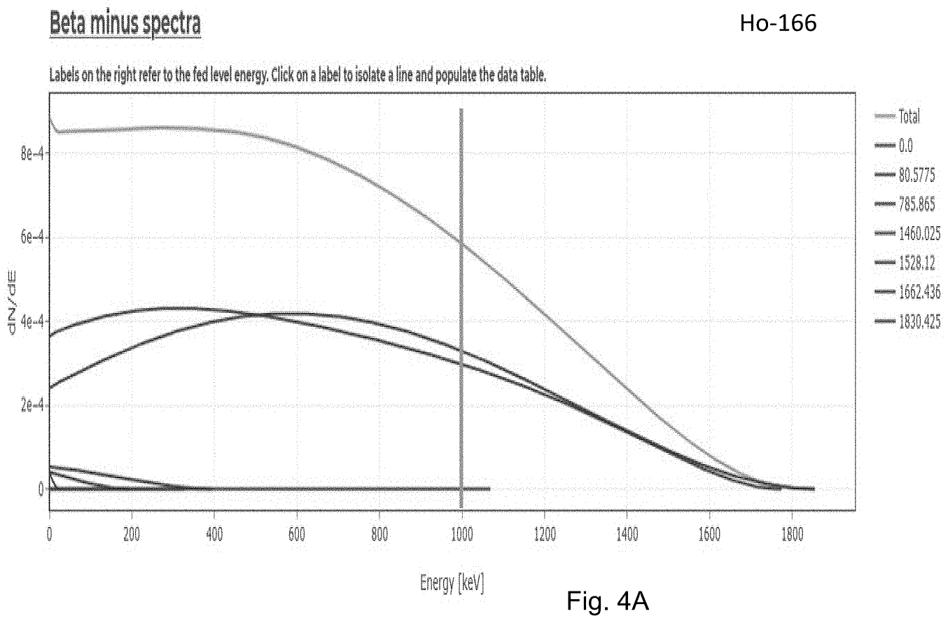

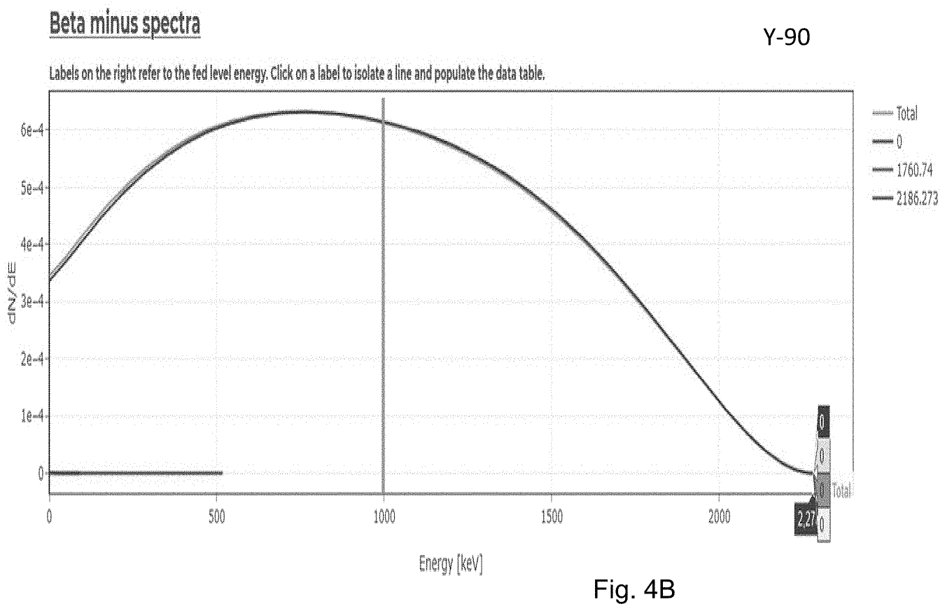

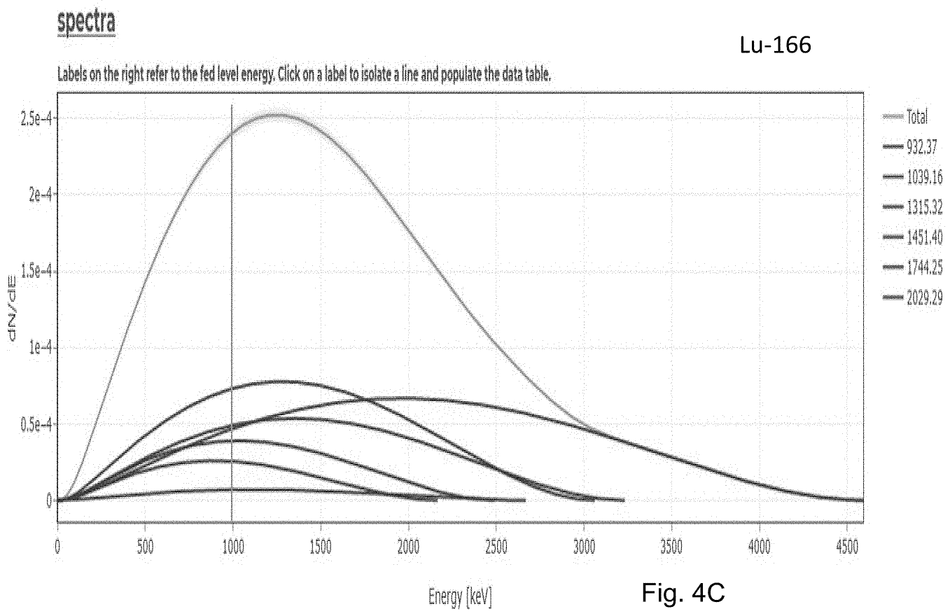

In the future some as yet un-isolated isotopes may be able to be created in the modern accelerators or reactors which having known decay schemes further enhancing and optimizing the ability to build a wearable patch and give patient specific optimal dose delivery. One example is Lu-166 which decays via a very high energy electron emission at 4.5 MeV approximately. As depicted in FIGS. 4A, 4B, and 4C, if we compare Ho-166 to Y-90 and Lu-166 in terms of decay spectra of electrons one can see how an isotope such as Lu-166, as it is commercially available, could enable continued evolution of this approach of optimizing and mixing isotopes. FIG. 4 shows the decay spectra from Ho-166, Y-90 and Lu-166 all aligned at 1 MeV energy to show the relative differences of the electrons emitted from each. Were Lu-166 available to measure the percent depth dose one could reasonably expect it to penetrate deeper and perhaps higher even than 6 MeV electrons depth dose. This can also be a convenient approach to have one isotope which excess depth dose range and then utilize the disclosed bolus thickness to customize it to a given patient's lesion depth.

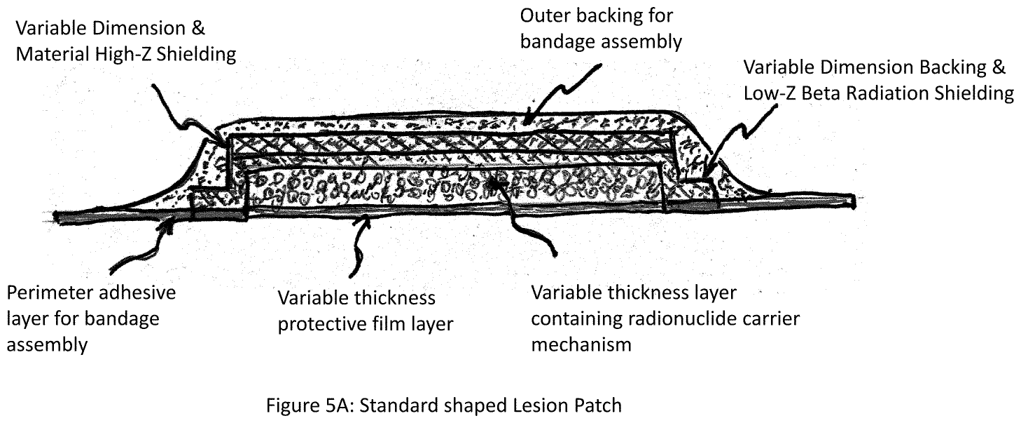

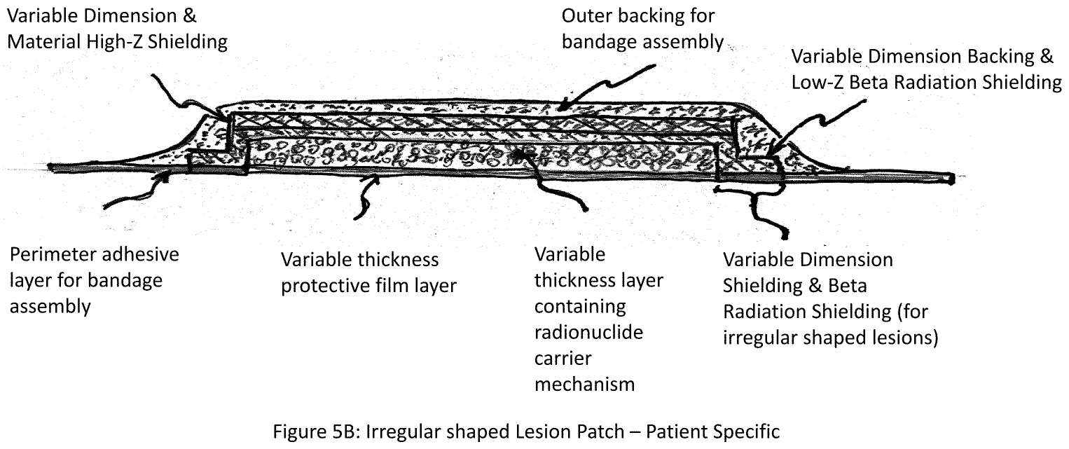

Another aspect of the invention is to disclose optimal use of radionuclides to fabricate a temporary covering or patch of materials containing sufficient radionuclide(s) also comprising a mixture of nuclides, with suitable decay schemes to mimic and closely resemble the dosimetry seen and used regularly with external beam electron and keV photon dosimetry. As depicted in FIGS. 5A and 5B, the cumulative, radio-biologically equivalent dose scheme of such an external beam radiotherapy device in cutaneous lesions is thus being approximated by a surface source of nuclides imbedded into the disclosed carrier mechanism forming the active portion of a covering or patch that would be worn for a long enough period to allow a curative prescribed dose to be deposited into the cutaneous lesions. Dose schemes including fractionation with a small number of sessions could also be implemented and the corresponding changes to the nuclide patch loading and design considered for a series of treatments which would be tailored such that they are radio-biologically equivalent to a 50 Gy total dose with 2 Gy per fraction in 25 fractions as is standard fractionation from an external beam source, for example. In an illustrative example using Palladium-103 and Iodine-125 in some mixture ratio with enough combined activity to provide an accelerated dose fractionation scheme that reduces the number of treatments from 30 down to 5, 3, or even a single treatment.

FIG. 5A depicts a cross section of the radionuclide patch design which allows dosimetry optimization and a convenient dose delivery mechanism. The patch is designed to have an adhesive layer in contact with the skin and around the perimeter of a standard set of shapes such as circles, ellipsoids, or rectangles, or be custom shaped for a given body site such as across the cheeks and nose in one large area patch as depicted in FIG. 5B.

As depicted in FIGS. 5A-5B, inside the patch is a radionuclide carrier mechanism which contains the mixture of radionuclides chosen individually or in a specific mixture for a given lesion which is variable in thickness depending upon the desired dosimetry. It has a minimal protective film layer between the radionuclide carrier mechanism and the skin which itself may be varied in dimension depending upon the energy of the decay particles coming from the radio nuclides. The protective film layer can serve as a bolus material should the electron energies of a given isotope become high enough to require it for assuring an optimum prescription at a given depth, similar to bolus used routinely with high energy electron beams from linear accelerators. Bolus can also be used for tailoring the highest dose of a gamma ray emitter as well for consideration of different fractionation schemes, for example for bulkier lesions with deeper aspects.

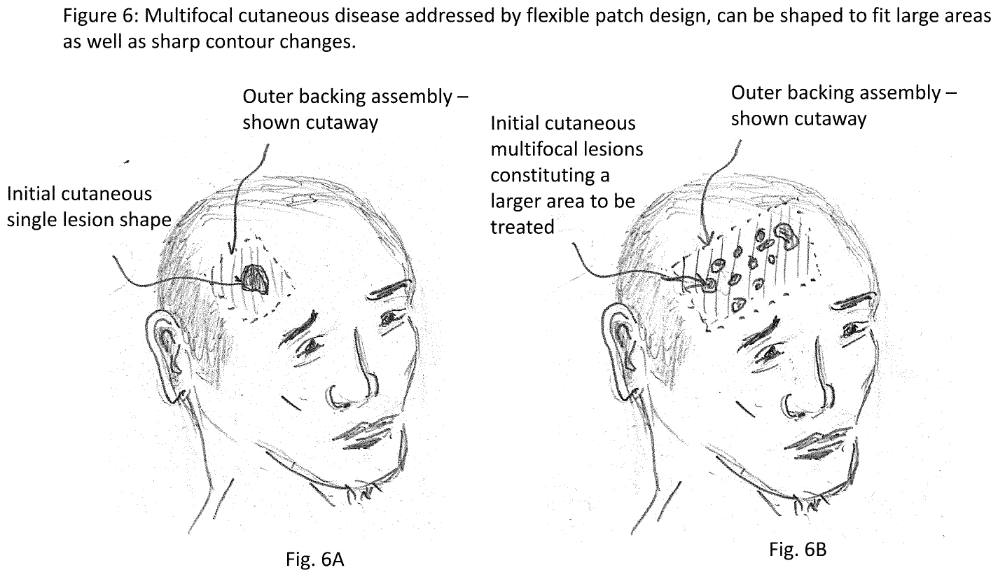

As depicted in FIGS. 6A-6B, in another embodiment, the invention would accommodate a larger area of multifocal disease as is common in many countries with high patient volumes of skin cancers of the face, nose, head, and ears and is quite challenging to treat effectively with an acceptable cosmetic outcome. The flexible nature of the patch and the supporting materials show allow for it to bend and take the contour of the patient's skin in challenging areas.

In the present invention, experience in radiation oncology and physics informs one skilled in the art that available medical isotopes placed within a patch type device can create a dosimetrically suitable alternative to using a linear accelerator or X-ray tube-based skin cancer treating device. Future developments of suitable candidate isotopes such as Lu-166 could also be utilized in the disclosed cutaneous lesion radionuclide patch.

While there has been shown a preferred embodiment of the present invention, it is to be understood that certain changes may be made in the forms and arrangement of the elements of radionuclide isotopes available for treatment of cutaneous lesions of the human body without departing from the underlying spirit, scope, and essential characteristics of the invention. The present embodiment is therefore, to be considered as merely illustrative and not restrictive, the scope of the invention being indicated by the claims rather than the foregoing description, and all changes which come within the meaning and range of equivalence of the claims are therefore intended to be embraced therein.

What is claimed is:

1. A radioactive patch comprising a carrier, a layer of a mixture of a radionuclide with a nonreactive adhesive agent coated thereon in the form of a tape, and a laminating layer;
wherein the patch comprises, a high Z shielding layer placed on the opposing side of the patch away from the patient tissue, and comprising at least one of: lead, tungsten, iron, silver, gold, platinum, copper, brass;
wherein the patch comprises, a low Z shielding layer of comprising at least one of: teflon, pma, pvc, lucite, boron carbide, graphite, carbon fiber, bakelite; and
wherein the radionuclide comprises at least one of: Y-90, Ho-166, LU-166, I-125, PD-103, LU-166, or any combination thereof.
